(19) United States
(12) Patent Application Publication
(10) Pub. No.: US 2018/0132810 A1
(43) Pub. Date: May 17, 2018

(54) SYSTEM FOR DETERMINING TISSUE DENSITY VALUES USING POLYCHROMATIC X-RAY ABSORPTIOMETRY

(71) Applicant: THE BOARD OF TRUSTEES OF THE LELAND STANFORD JUNIOR UNIVERSITY, Stanford, CA (US)

(72) Inventors: Luis de Sisternes, Stanford, CA (US); Daniel L. Rubin, Stanford, CA (US); Jan Liphardt, Stanford, CA (US)

(21) Appl. No.: 15/580,626

(22) PCT Filed: Jun. 8, 2016
(86) PCT No.: PCT/US16/36496

§ 371 Date: Dec. 7, 2017

Related U.S. Application Data
(60) Provisional application No. 62/173,006, filed on Jun. 9, 2015.

(51) Int. Cl. A61B 6/00; G06T 7/00

(52) U.S. Cl. CPC A61B 6/502; G06T 2207/30068; G06T 2207/10116; G06T 7/0012

(57) ABSTRACT

Provided are computer-implemented methods of determining tissue composition by polychromatic absorptiometry. The methods include acquiring a raw intensity image of a tissue comprising dense tissue and adipose tissue. The image is generated using a polychromatic electromagnetic radiation source. The methods further include directly measuring the proportion of dense tissue and adipose tissue for each pixel of the raw intensity image and assigning a value to each pixel based on the directly measured proportion of dense tissue and adipose tissue. The composition of the tissue is determined based on the assigned value of each pixel. Systems for practicing the methods are also provided.

### CROSS-REFERENCE TO RELATED APPLICATIONS

This application claims priority from U.S. Provisional Patent Application No. 62/173,006, filed Jun. 9, 2015, which is hereby incorporated by reference in its entirety.

### STATEMENT OF GOVERNMENTAL SUPPORT

None.

### REFERENCE TO SEQUENCE LISTING, COMPUTER PROGRAM, OR COMPACT DISK

None.

### BACKGROUND OF THE INVENTION

### Field of the Invention

The present invention relates to the fields of image analysis, and, more particularly, to mammography, e.g. full-digital mammography (FFDM), methods of the amount of dense breast tissue, and methods for correlating tissue density to cancer risk.

### Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

It is known that breast tissue is composed of fibrous and glandular (fibroglandular) and fatty tissue, where fibroglandular tissue radiologically appears dense on X-ray mammograms and fatty tissue appears lucent. The term breast density, also called mammographic density, has been used to refer to an estimate of the relative proportion of area that the fibroglandular tissue occupies in the breast tissue as presented in a mammogram.

Women with high mammographic density can have four- to six-times the risk of breast cancer relative to women with predominantly fatty breasts. This may be accounted for by an etiologic effect, which is reflected in the fact that breast cancers predominantly develop in the epithelial cells that line the ducts of the breast. High mammographic density, which reflects breast composition of predominantly fibrous and glandular tissue, may therefore indicate an increased likelihood of developing breast cancer.

Breast density has been assessed subjectively using various approaches including categorical scales, Visual Analogue Scales, and semi-automated threshold-based algorithms to describe breast composition in terms of mammographic density. One such subjective approach is the American College of Radiology (ACR) Breast Imaging Reporting and Data System (BI-RADS) breast composition assessment scale. This scale has been described in numerous studies relating the appearance of mammographic density to breast cancer risk. The ACR BI-RADS scale describes four categories of mammographic breast density and breast composition which radiologists are recommended to use in the evaluation of mammographic density. A shortcoming of the ACR BI-RADS scale for reporting breast density is that it is reader dependent—a subjective estimate of breast density that may be biased and not reliably reproducible.

### SPECIFIC PATENTS AND PUBLICATIONS

Shepherd et al., “Determining body composition using fan beam dual-energy x-ray absorptiometry,” U.S. Pat. No. 6,233,473, discloses methods for using dual-energy x-ray absorptiometry to determine whole body and regional composition. This system considers a dual-energy x-ray system, rather than a continuous spectrum. Shepherd et al. have been used to measure or estimate parameters such as bone mineral density (BMD). Further when pencil-beam systems are used for body composition measurements, the attenuation measurement for all the pixels are obtained by measuring the intensity of x-rays that travel along essentially parallel paths. However, when a system with a fan-shaped x-ray distribution is used, there are geometric and other factors that can complicate body fat computations and introduce inaccuracies.

Shepherd et al., “Novel use of single X-ray absorptiometry for measuring breast density,” Technol Cancer Res Treat. 2005 April; 4(2):173-82, discloses an automated method for measuring breast composition, called Breast Compositional Density measured using single X-ray absorptiometry techniques, or BD_{(SXA)}. BD_{(SXA)} measures breast compositional density by comparing the opacity on the mammogram to two reference standards imaged with each breast. SXA is described as solves for breast composition by comparing the gray scale of a pixel in the mammogram to the gray scale of fat and lean references in a phantom compressed to the same thickness as the breast and imaged with the breast.

### BRIEF SUMMARY

The present method comprises quantifying the density of a volume of soft tissue having varied components of different density. This method is applicable to medical digital image analysis. It is applicable to breast tissue, but may also be applied to skin and other organs that are digitally imaged using tissue-penetrating light, e.g. polychromic X-ray. The exemplified application of the present method is based on a digital mammogram, such as a full digital mammogram (FFDM), which can be prepared using commercially available X-ray equipment and software. The present methods may be carried during mammography or by analyzing a previously prepared mammogram and the accompanying data acquired in a typical mammogram screening session. The mammogram image and accompanying metadata (discussed below) are analyzed pixel-by-pixel according to the presently described methods, and the pixel data are used to create an enhanced image and quantitate results of total dense volume and dense-to-adipose tissue in the image (See ref no. 108 in FIG. 1 and FIG. 4)

Using the present methods, a mammogram under study is processed using a physical model of polychromatic breast tissue absorption and a pixel-based correction factor derived from total breast thickness and X-ray source characteristics (“breast thickness estimation,” ref. no. 105 in FIG. 1, and “source spectrum calibration”, ref no. 106 in FIG. 1) to account for different apparent density data generated by a polychromic X-ray source. That is, the method uses an internal reference derived from the raw image intensity and metadata, rather than an extrinsic construction included in the image (i.e., a phantom).

Further, the method does not use effective linear attenuation coefficients (singular constant value) derived from the imaging properties or a singular value of breast thickness. Instead, the contribution of a continuous energy spectrum and the dependence of attenuation coefficients with energy are considered, as well as a pixel-based value for breast thickness, estimated from an adipose-equivalent image (see ref no. 104 in FIG. 1).

The method further uses a constrained linear equation to arrive at total breast thickness at each particular pixel location and to obtain a proportion of adipose and dense tissue in the breast.

The presently disclosed method is similar to the Cumulus approach in that both aim to differentiate breast density from surrounding fatty tissue. Unlike the Cumulus approach, which “masks” the image into dense and non-dense tissue, the present method provides direct measurements of:

(a) breast-to-adipose tissue ratio (same as dense-to-adipose tissue);

(b) total breast volume; and

(c) dense tissue volume, in a continuous scale at each image pixel.

The method also measures breast density relative to breast thickness at each particular location in the breast (specifically at each pixel in the mammogram image), employing the adipose-equivalent image (FIG. 1, ref. no. 104). This approach is different than previous approaches in that the estimated adipose-equivalent image obviates the requirement of the presence of a phantom in the mammogram for calibration purposes. It also differs from previous approaches that do not require a phantom, in that the adipose-equivalent image allows a pixel-by-pixel calibration dependent on breast thickness, instead of considering a singular intensity value for calibration throughout the mammogram, e.g., obtained by considering pixels surrounding the edge of the breast in the mammogram.

The above measurements are also correlated to reference data correlated to breast cancer and used in evaluating breast cancer risk.

Aspects of the present disclosure include computer-implemented methods of determining tissue composition by polychromatic absorptiometry. The methods include acquiring a raw intensity image of a tissue (e.g., breast tissue) comprising dense tissue and adipose tissue. The image is generated using a polychromatic electromagnetic radiation source. The methods further include directly measuring the proportion of dense tissue and adipose tissue for each pixel of the raw intensity image and assigning a value to each pixel based on the directly measured proportion of dense tissue and adipose tissue. The composition of the tissue is determined based on the assigned value of each pixel.

In some embodiments, the methods further include, prior to aquiring the raw intensity image, irradiating the tissue using the polychromatic electromagnetic radiation source to generate the raw intensity image. In certain aspects, the polychromatic electromagnetic radiation source is a polychromatic X-ray source.

According to certain embodiments, the methods further include displaying the tissue composition in the form of a dense volume image, a ratio of dense-to-adipose tissue image, or both.

In certain aspects, the methods further include determining the risk of cancer in the tissue based on the determined tissue composition. For example, when the tissue is breast tissue, the methods may include determining the risk of breast cancer based on the determined breast composition.

Systems for practicing the methods of the present disclosure are also provided. In certain aspects, provided is a polychromatic absorptiometry system, which system includes a processor and a non-transitory computer readable medium. The non-transitory computer readable medium includes instructions that cause the processor to acquire a raw intensity image of a tissue (e.g., breast tissue) that includes dense tissue and adipose tissue, where the image is generated using a polychromatic electromagnetic radiation source. The instructions further cause the processor to directly measure the proportion of dense tissue and adipose tissue for each pixel of the raw intensity image, assign a value to each pixel based on the directly measured proportion of dense tissue and adipose tissue, and determine tissue composition based on the assigned value of each pixel.

According to certain embodiments, the system further includes a polychromatic electromagnetic radiation source and a detector adapted to generate the raw intensity image. The polychromatic electromagnetic radiation source may be, e.g., a polychromatic X-ray source.

The systems of the present disclosure may include a display (e.g., an LCD, LED, or other suitable display). In certain aspects, the instructions further cause the processor to display the tissue composition in the form of a dense volume image, a ratio of dense-to-adipose tissue image, or both.

According to certain embodiments, the instructions may further cause the processor to determine the risk of cancer in the tissue.

In certain aspects, the present invention comprises a computer-implemented method of determining areas of tissue density and composition by use of polychromatic absorptiometry, comprising: acquiring a raw, digital intensity image of a tissue comprising different areas of tissue density, wherein less dense tissue comprises adipose tissue, and wherein the image is generated using a polychromatic electromagnetic radiation source; correcting attenuation effects on density associated with energy differences within the polychromatic electromagnetic radiation source; directly measuring the proportion of dense tissue and adipose tissue for each pixel of the raw intensity image using an adipose-equivalent intensity estimation; assigning a value to each pixel based on the directly measured proportion of dense tissue and adipose tissue; and determining tissue composition based on the assigned value of each pixel.

In certain aspects, the present invention comprises a method as described above, further comprising, prior to acquiring the raw intensity image, a step of irradiating tissue in vivo using the polychromatic electromagnetic radiation source to generate the raw intensity image.

In certain aspects, the present invention comprises a method as described in one or more of the paragraphs above, wherein the polychromatic electromagnetic radiation source is a polychromatic X-ray source.

In certain aspects, the present invention comprises a method as described in one or more of the paragraphs above, further comprising displaying the tissue composition in the form of a dense volume image, a ratio of dense-to-adipose tissue image, or both.

In certain aspects, the present invention comprises a method as described in one or more of the paragraphs above, further comprising determining the risk of cancer in the tissue based on the determined tissue composition.

In certain aspects, the present invention comprises a method as described in one or more of the paragraphs above, wherein the tissue is breast tissue.

In certain aspects, the present invention comprises a method as described in one or more of the paragraphs above wherein the raw, digital intensity image is an X-ray mammogram image, the step of determining tissue composition comprises producing a quantification of dense volume of an imaged breast and also producing a ratio of dense to adipose tissue of the imaged breast.

In certain aspects, the present invention comprises a method as described in one or more of the paragraphs above, further the step of calculating a risk of developing breast cancer in the imaged breast, based on quantification of dense volume in the imaged breast and the ratio of dense to adipose tissue in the imaged breast.

In certain aspects, the present invention comprises a polychromatic absorptiometry system, comprising a processor; a non-transitory computer readable medium comprising instructions that cause the processor to: acquire a raw intensity image of a tissue comprising dense tissue and adipose tissue, wherein the image is digital and generated using a polychromatic electromagnetic radiation source; directly measure the proportion of dense tissue and adipose tissue for each pixel of the raw intensity image, wherein density is calculated using a correction for energy variations within the polychromatic electromagnetic radiation source; assign a value to each pixel based on the directly measured proportion of dense tissue and adipose tissue; and determine tissue composition based on the assigned value of each pixel.

In certain aspects, the present invention comprises a system as above, further comprising a polychromatic electromagnetic radiation source and a detector adapted to generate the raw intensity image.

In certain aspects, the present invention comprises a system as described in one or more of the paragraphs above, wherein the polychromatic electromagnetic radiation source is a polychromatic X-ray source.

In certain aspects, the present invention comprises a system as described in one or more of the paragraphs above, further comprising a display that graphically displays areas of density within the tissue.

In certain aspects, the present invention comprises a system as described in one or more of the paragraphs above, wherein the instructions further cause the processor to display the tissue composition in the form of a dense volume image, a ratio of dense-to-adipose tissue image, or both.

In certain aspects, the present invention comprises a system as described in one or more of the paragraphs above, wherein the instructions further cause the processor to determine the risk of cancer in the tissue.

### BRIEF DESCRIPTION OF THE DRAWINGS

FIG. 1 is a flow diagram of a polychromatic X-ray absorptiometry (PXA) method according to one embodiment of the present disclosure. Digital mammography Screening 101 (input) produces raw image intensity (figure ref. no) 102 and metadata 103. This is used to produce an adipose equivalent intensity estimation 104 and also inputs to the estimation of breast composition based on tissue attenuation properties, shown at 107. The adipose equivalent intensity estimation 104 is used to produce a breast thickness estimation (BTE) 105. The BTE 105 also receives input from detector calibration 105a which receives input from metadata 103 and adipose equivalent intensity estimation 104. As can be seen, detector calibration 105a inputs to the BTE 105 and to the “Estimation of breast composition based on tissue attenuation properties,” which, as shown is carried out on a computer system 107. As shown, computer system 107 receives input from the raw image intensity 102, the BTE 105, the detector calibration and “source spectrum calibration” 106. Blocks 103, 102 (including DICOM data), 105, 105a and 106 provide data to the estimation 107 done by the computer. This part of the system is shown as the general “processing block” (figure ref nos. 102-107) in FIG. 1. The Output block, following the “processing block” is shown as blocks 108 (dense volume), 109 (ratio dense/adipose tissue) and the Quantification associated with breast cancer risk, shown at 110, and receiving inputs from image data 108 and 109.

FIG. 2 shows (see panels A, B, C, D) images relating to adipose-equivalent intensity estimation. The images are shown in terms of absorption (the negative logarithm of the recorded intensity). Panel A: Original absorption image −log(I_{(x,y)}). Panel B: Absorption image with candidate locations of mainly adipose tissue indicated in green. Panel C: Absorption image with refined candidate locations indicated in blue. Panel D: Estimated adipose-equivalent absorption image −log (_{\hat{F}(x, y)}). The original false colors green and blue are indicated by 202 and 204, respectively, and appearing in Panels B and C.

FIG. 3 shows (see panels A, B, C) the results of preliminary work using a physical phantom. Panel A (phantom mammogram): Digital mammogram of the fabricated phantom. Collagen to butter concentration is indicated for each row. The automatically detected location of the wells is outlined. Panel B (Estimated collagen ratio): Estimated collagen density ratio image for the pixels in the detected wells. Panel C: Estimated average collagen density throughout each well displayed against the actual fabricated density. The values in the box indicate the Pearson's correlation coefficient and its computed p-value.

FIG. 4, Panel A (left, control example) and Panel B (right, case example), provides examples of percentage of dense-to-adipose tissue (PD2A) images generated by the PXA method according to one embodiment of the present disclosure for case (right) and control (left) mammograms. The red boundary in the images indicates the extent where the PD2A images where computed.

### DETAILED DESCRIPTION

Provided are computer-implemented methods of determining tissue composition by polychromatic absorptiometry. The methods include acquiring a raw intensity image of a tissue comprising dense tissue and adipose tissue. The image is generated using a polychromatic electromagnetic radiation source. The methods further include directly measuring the proportion of dense tissue and adipose tissue for each pixel of the raw intensity image and assigning a value to each pixel based on the directly measured proportion of dense tissue and adipose tissue. The composition of the tissue is determined based on the assigned value of each pixel. Systems for practicing the methods are also provided.

Before the methods and systems of the present disclosure are described in greater detail, it is to be understood that the methods and systems are not limited to particular embodiments described, as such may, of course, vary. It is also to be understood that the terminology used herein is for the purpose of describing particular embodiments only, and is not intended to be limiting, since the scope of the methods and systems will be limited only by the appended claims.

Ranges

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and systems. The upper and lower limits of these smaller ranges may independently be included in the smaller ranges and are also encompassed within the methods and systems, subject to any specifically excluded limit in the stated range. Where the stated range includes one or both of the limits, ranges excluding either or both of those included limits are also included in the methods and systems.

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

The term “computer-implemented,” as described herein, refers to the use of a special purpose, or general purpose computer, comprising a processor, read and write functions, and a display operating together, as is known in the art. Implementation comprises software.

The term “raw intensity image,” as described herein, refers to a digital image as produced by an imaging device such as a commercially available X-ray machine for medical use; this term is further explained in connection with the discussion of the DICOM standard.

The term “a polychromatic electromagnetic radiation source,” as described herein, produces radiation that contains an essentially continuous range of energies (and therefore wave lengths), for example, a tube with a molybdenum anode can be used with about 30 000 volts (30 kV), giving a range of X-ray energies of about 15-30 keV; see for details http colon-slash-slash-www(dot)arpansa(dot)gov.au/radiationprotection/basics/xrays.cfm, which details properties of different X-ray properties and illustrates a sample calculated X-ray spectrum, with a tungsten target and a 13° angle. Many of these photons are “characteristic radiation” of a specific energy determined by the atomic structure of the target material (Mo-K radiation). This radiation source is to be contrasted from the source as used in dual energy X-ray absorptiometry, where emitted X-ray in two narrow beams that are scanned across the patient.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and systems belong. Although any methods and systems similar or equivalent to those described herein can also be used in the practice or testing of the methods and systems, representative illustrative methods and systems are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and systems are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

It is appreciated that certain features of the methods and systems, which are, for clarity, described in the context of separate embodiments, may also be provided in combination in a single embodiment. Conversely, various features of the methods and systems, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions/kits. In addition, all sub-combinations listed in the embodiments describing such variables are also specifically embraced by the present methods and systems and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

### Methods

As summarized above, aspects of the present disclosure include computer-implemented methods of determining tissue composition by polychromatic absorptiometry.

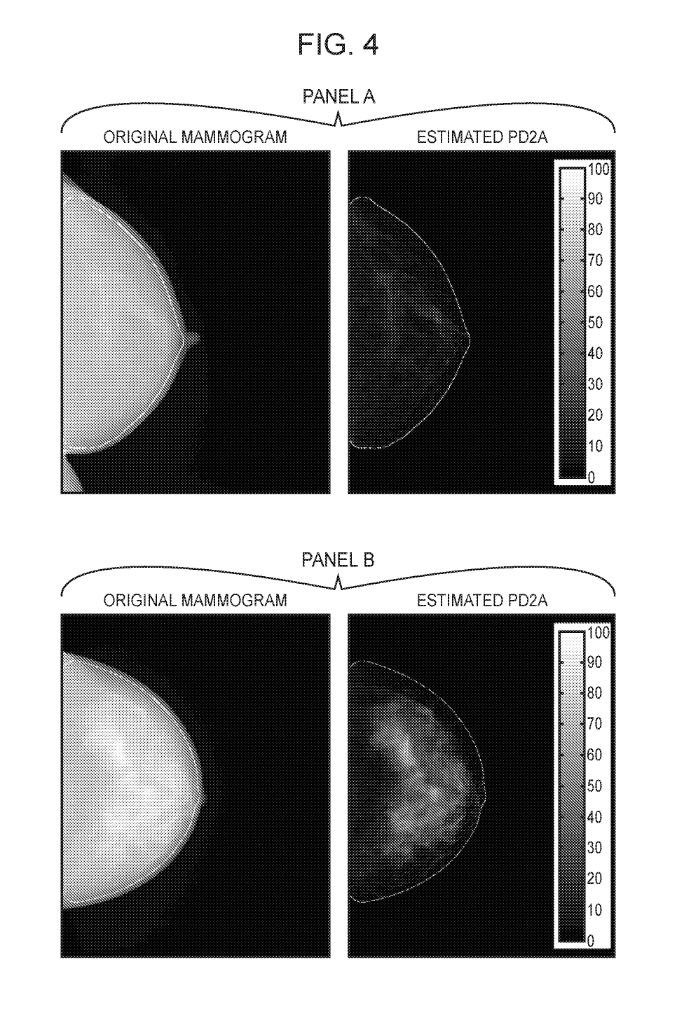

The present method includes quantifying the density of a volume of soft tissue having varied components of different density within a defined area. This is applicable to breast tissue, but may also be applied to skin and other organs that are digitally imaged using tissue-penetrating light. The exemplified application of the present methods uses a digital mammogram, such as a full digital mammogram (FFDM), which can be prepared using commercially available equipment and software. The method may be carried out during mammography or by analyzing a previously prepared mammogram and the accompanying data acquired in a typical mammogram screening session. The mammogram image and accompanying metadata is analyzed pixel-by-pixel according to the presently described methods, and the pixel data are used to create an informed image and a quantitative result of dense-to adipose tissue in the image (see FIG. 4).

According to the present methods, a mammogram under study is processed using a correction factor (“source spectrum calibration”) to account for different apparent density data generated by a polychromic electromagnetic radiation (e.g., X-ray) source. That is, the method uses an internal reference method, rather than an extrinsic construction included in the image (i.e., a phantom).

Further, the method may use energy-dependent linear attenuation coefficients derived from the imaging properties and common knowledge, and a pixel-dependent value of breast thickness.

The method may further use a constrained linear equation to arrive at total breast thickness at each particular pixel location and to obtain a proportion of adipose and dense tissue at each pixel.

The methods of the present disclosure are similar to the Cumulus approach in terms that both aim to differentiate breast density from surrounding fatty tissue. Unlike the Cumulus approach, which “masks” the image into dense and non-dense tissue (categorizing each image pixel in two divided categories: either mainly dense or mainly adipose tissue), the present method provides direct measurements of: dense-to-adipose tissue ratio; total breast volume; and dense tissue volume, in a continuous scale at each image pixel. The “Cumulus” method is described in McCormack V A, dos Santos S I. “Breast density and parenchymal patterns as markers of breast cancer risk: a meta-analysis.” Cancer Epidemiol. Bio. Prey. 2006; 15:1159-69.

The methods may also measure breast density relative to breast thickness at each particular location, employing the adipose-equivalent image, instead of the value of a single fatty tissue pixel used in previous methods.

The above measurements may be correlated to reference data correlated to breast cancer and are useful in evaluating breast cancer risk.

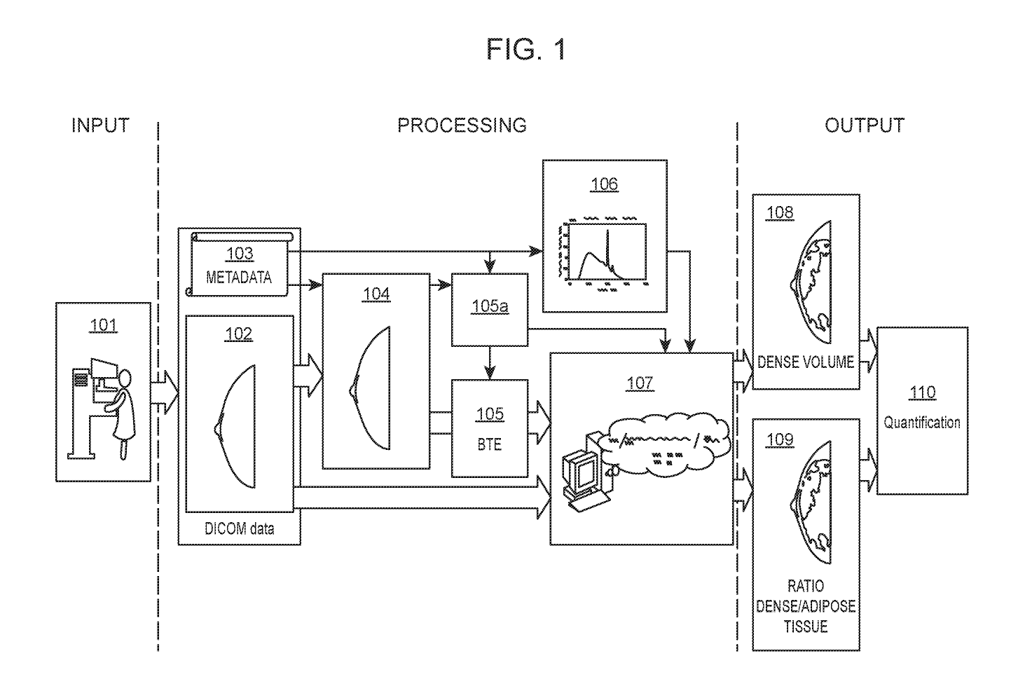

An overview of a method according to one embodiment of the present disclosure is shown in FIG. 1. As shown there, a digital mammogram is obtained from a woman being screened or diagnosed, shown at 101. This step produces a digital image (mammogram), termed as a raw intensity image, i.e., as directly received from the X-ray machine (this may include digitizing a film image). This image 102 will be accompanied by metadata, shown at 103. The legend “DICOM data” refers to a standard data protocol that contains metadata such as the X-ray source used, the image acquisition time, etc. See, the DICOM (Digital Imaging and Communication in Medicine) web site at http colon-slash-slash-medical.nema (dot) org web site for further details.

The Digital Imaging and Communications in Medicine (DICOM) standard was created by the National Electrical Manufacturers Association (NEMA) to aid the distribution and viewing of medical images, such as CT scans, MRIs, and ultrasound. Part 10 of the standard describes a file format for the distribution of images. This format is an extension of the older NEMA standard. Most people refer to image files which are compliant with Part 10 of the DICOM standard as DICOM format files. A single DICOM file contains both a header (which stores information about the patient's name, the type of scan, image dimensions, etc.), as well as all of the raw image data as defined (which can contain information in three dimensions). This is different from the popular Analyze format, which stores the image data in one file (.img) and the header data in another file (.hdr). Another difference between DICOM and Analyze is that the DICOM image data can be compressed (encapsulated) to reduce the image size. Files can be compressed using lossy or lossless variants of the JPEG format, as well as a lossless Run-Length Encoding format (which is identical to the packed-bits compression found in some TIFF format images). (see, http colon-slash-slash-www-dot-mccauslandcenter.sc-dot-edu/mricro/dicom/

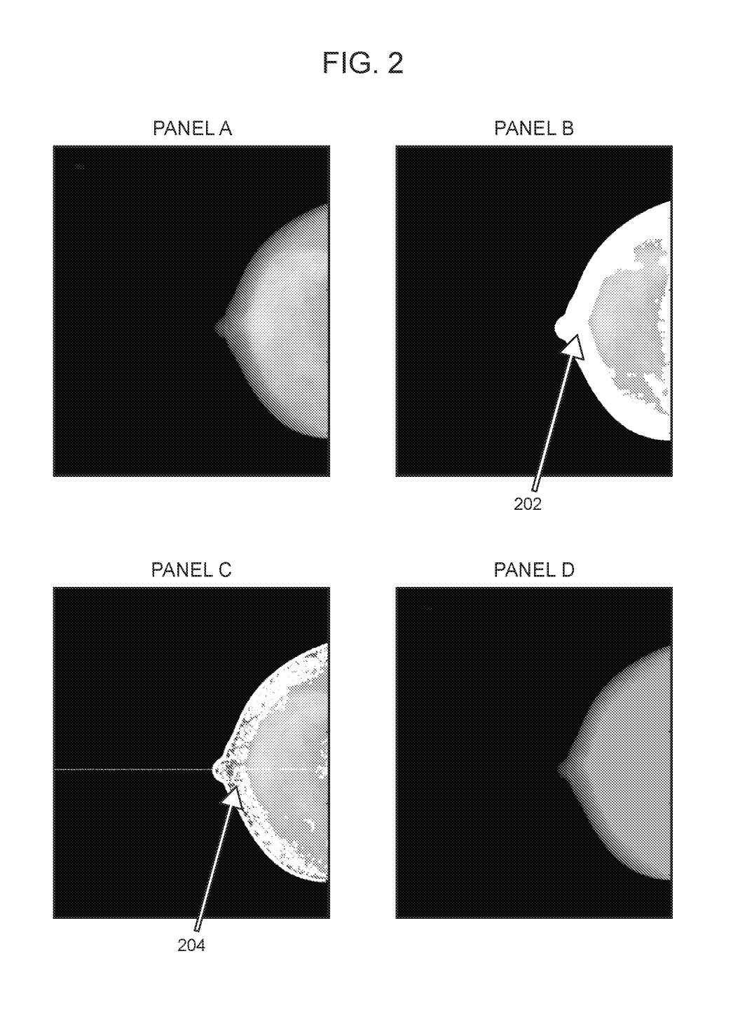

As shown at 104, the raw intensity image is analyzed to obtain an “adipose-equivalent intensity estimation.” The adipose-equivalent intensity estimation used metadata and image 102. Its calculation is explained in connection with FIG. 2. The adipose-equivalent intensity estimation is used to estimate breast thickness and to obtain a detector calibration. The detector calibration corrects possible internal normalization made by the mammography system X-ray detector or digitizer. As shown at 106, the metadata is used to obtain a source spectrum calibration. The X-ray used to generate the raw image intensity is, as shown, a range of photon energies, wherein the continuous range of photon energies produce a variable number of photons. For example, photon energies may range from about 20-100 keV, an a larger number of photons at about 40 keV from braking radiation. A sharp peak at about 60 keV may be present from a tungsten characteristic X-ray. The different energies will have different wavelengths and frequencies. As discussed below, the present invention comprises the use of a correction factor for various pixel intensities that will be used to calculate tissue density.

Using (1) breast thickness estimation, (2) the detector calibration and (3) the source spectrum calibration, an estimate of breast composition is calculated using the computer considering a physical model of polychromatic absorptiometry in adipose and dense breast tissue, as shown at 107. As indicated, this is based on tissue attenuation properties determined above. The breast thickness estimation (comprising a pixel-by-pixel description of total breast thickness), the correction factors derived from the detector calibration, and the correction factor derived from the source spectrum calibration (intensities at each energy range) are used in the expression describing the physical model to solve the percentage amount of dense and adipose tissue from the total breast thickness at each pixel in the mammogram (see equation (2) below).

Finally, as shown at 110, the estimate of breast composition is used to generate images showing dense volume and a ratio of dense/to adipose tissue in an image. In addition, the data may be associated with breast cancer risk.

According to this embodiment, raw intensity image data and metadata acquired in a typical digital mammography screening session is processed automatically to generate a pixel-by-pixel estimation of the volumetric density composition within the breast. The estimated breast composition can be displayed in the form of dense volume and ratio of dense-to-adipose tissue images, which can be further processed to generate quantifications associated with breast cancer risk.

According to certain embodiments, a computer-implemented method of determining tissue composition by polychromatic absorptiometry includes acquiring a raw intensity image of a tissue comprising dense tissue and adipose tissue, where the image is generated using a polychromatic electromagnetic radiation source. The method further includes directly measuring the proportion of dense tissue and adipose tissue for each pixel of the raw intensity image, assigning a value to each pixel based on the directly measured proportion of dense tissue and adipose tissue, and determining tissue composition based on the assigned value of each pixel.

In some embodiments, the methods further include, prior to acquiring the raw intensity image, irradiating the tissue using the polychromatic electromagnetic radiation source to generate the raw intensity image. In certain aspects, the polychromatic electromagnetic radiation source is a polychromatic X-ray source.

According to certain embodiments, the methods further include displaying the tissue composition in the form of a dense volume image, a ratio of dense-to-adipose tissue image, or both.

In certain aspects, the methods further include determining the risk of cancer in the tissue based on the determined tissue composition. For example, when the tissue is breast tissue, the methods may include determining the risk of breast cancer based on the determined breast composition. The determined breast composition may be given a score, and determining the risk of breast cancer may be based on the score. Alternatively, or additionally, the risk of breast cancer may be based on images displayed to a practitioner (e.g., a radiologist), such as a dense volume image, a ratio of dense-to-adipose tissue image, or both. Visualization of abnormalities in such images is improved according to the methods/systems of the present disclosure as compared to existing approaches. In certain aspects, the risk of breast cancer in an individual is determined by inspection of a dense volume image, a ratio of dense-to-adipose tissue image, or both, produced using the methods of the present disclosure.

Approaches for determining tissue composition according to certain embodiments of the present disclosure will now be described in the context of determining breast composition.

According to the Beer-Lambert law, the intensity image I(x, y) recorded at the detector in a full-digital mammography system follows the expression:

$$I ( x , y ) = H ( {\int }_{0}^{{ɛ}_{max}} S ( ɛ ) {e}^{- {\int }_{0}^{L ( x , y )} μ ( x , y , z ; ɛ ) \partial z} \partial ɛ / {\int }_{0}^{{ɛ}_{max}} S ( ɛ ) \partial ɛ ) , \qquad ( 1 )$$

where ε indicates the X-ray energy parameter, covering a range from 0 to εₘₐₓ (highest energy in the source spectrum); S(ε) describes the energy-dependent intensity of the X-ray source; μ(x, y, z; ε) describes the attenuation coefficient of the breast sample at each volumetric position, which is also energy dependent; and H(·) is the particular internal normalization function of the system's detector. Assuming that breast is mainly composed of adipose and dense tissue, the proportion of each of these tissue types at a particular pixel, ratio_{ad}(x, y) and ratio_{den}(x, y), respectively, can be then estimated from the raw recorded intensity values by solving the expression:

$${I ( x , y ) = {c}_{1} ( ( {\int }_{0}^{{ɛ}_{max}} \hat{S} ( ɛ ) {e}^{- ( {μ}_{ad} ( ɛ ) {ratio}_{ad} ( x , y ) + {μ}_{den} ( ɛ ) {ratio}_{den} ( x , y ) ) \hat{L} ( x , y )} \partial ɛ / {\int }_{0}^{{ɛ}_{max}} \hat{S} ( ɛ ) \partial ɛ ) + {c}_{2} )}_{\begin{matrix}{ratio}_{den} ( x , y ) = 1 - {ratio}_{ad} ( x , y ) \\ 0 \leq {ratio}_{den} ( x , y ) \leq 1\end{matrix}} , \qquad ( 2 )$$

where μ_{ad}(ε) and μ_{den}(ε) indicate the attenuation coefficients of adipose and dense tissue, respectively, Ŝ(ε) indicates an estimation of the X-ray source spectrum and \hat{L}(x, y) is an estimation of total breast thickness at each pixel position. The coefficients c₁ and c₂ are related to the detector internal normalization function. This normalization function can be known or measured directly, but in certain aspects, it is assumed to be linear for simplicity, and the coefficients are computed as part as the detector calibration in an intensity image normalization process, as explained herein below.

A solution for the expression in equation (2), solving for ratio_{ad} (x, y) and ratio_{den}(x, y), can be found using a constrained non-linear optimization technique, where the energy attenuation coefficients of adipose and dense tissue are known, as reported in previous literature. Total breast thickness at each particular pixel location can be estimated from the recorded intensity values using image processing techniques. Although different methods can be applied for breast thickness estimation, the approach employed in the Examples section herein is described. This includes an estimation of an adipose-equivalent intensity image and a later correction using the normalization coefficients c₁ and c₂.

According to certain embodiments, determining breast composition includes estimating adipose-equivalent intensity. In certain aspects, an estimation of an adipose-equivalent intensity image \hat{F}(x, y) is generated by processing the recorded intensity image I(x, y). \hat{F}(x, y) corresponds to the estimated intensity recorded in the detector with a sample of the same thickness characteristics as the one imaged, but composed entirely by adipose tissue. Assuming that breast tissue thickness is not expected to decrease from nipple to chest wall, higher intensity values in this direction should be observed where adipose tissue is the most predominant, since adipose is the least-absorbing tissue type in breast. A set of candidate locations mainly containing adipose tissue within the image I(x, y) are selected by considering those with values that are monotonically decreasing from nipple to chest wall in each horizontal line. These locations are further refined by eliminating those which intensity does not follow a monotonically decreasing function in the vertical direction from top of the image to the nipple horizontal location, and form bottom of the image to nipple horizontal location, respectively, considering an assumption that thickness should be at its highest in the vertical direction at nipple level.

The image \hat{F}(x, y) is generated by fitting a surface to the values in the refined candidate locations, followed by a morphological opening with a disk kernel of one tenth of the horizontal sample extent. FIG. 2 displays an example of this estimation, with the images shown in terms of absorption, that is −log(I_{(x, y)}) in Panels A-C, and −log (_{\hat{F}(x, y)}) in Panel D, so differences can be better appreciated. The initial candidate locations are displayed in Panel B with green markings (see ref no. 202) and the result of their refinement is displayed in Panel C with blue markings (204).

In certain aspects, determining breast composition includes estimating system source spectrum (source spectrum calibration). According to certain embodiments, the X-ray source spectra is estimated as recorded directly from the mammography system when a subject is not present (on air) in a calibration process at the particular system settings, or generated using simulation techniques. For example, the X-ray source spectra indicated in equation (2) may be simulated considering the acquisition system characteristics recorded in the DICOM metadata.

According to certain embodiments, determining breast composition includes normalizing an intensity image. In certain aspects, the normalization coefficients c₁ and c₂ in equation (2) are computed by considering the estimated breast thickness, the statistics of the intensity recorded in air, and the maximum sample thickness recorded in the image DICOM metadata. Considering equation (1) and the linear attenuation coefficient in air (μₐᵢᵣ), a solution for c₁ and c₂ is computed by:

$${c}_{1} = \frac{min ( \hat{F} ( x , y ) ) - {\hat{I}}_{Air}}{\begin{matrix}( {\int }_{0}^{{ɛ}_{max}} \hat{S} ( ɛ ) {e}^{- {μ}_{ad} ( ɛ ) {L}_{max}} \partial ɛ / {\int }_{0}^{{ɛ}_{max}} \hat{S} ( ɛ ) \partial ɛ ) - \\ ( {\int }_{0}^{{ɛ}_{max}} \hat{S} ( ɛ ) {e}^{- {μ}_{air} ( ɛ ) {L}_{s - d}} \partial ɛ / {\int }_{0}^{{ɛ}_{max}} \hat{S} ( ɛ ) \partial ɛ )\end{matrix}} , \\ {c}_{2} = \frac{{\hat{I}}_{Air}}{{c}_{1}} - ( {\int }_{0}^{{ɛ}_{max}} \hat{S} ( ɛ ) {e}^{- {μ}_{air} ( ɛ ) {L}_{s - d}} \partial ɛ / {\int }_{0}^{{ɛ}_{max}} \hat{S} ( ɛ ) \partial ɛ ) \qquad ( 3 )$$

where ÎAir is the intensity recorded in the detector in air, which may be estimated by the median of the values of I(x, y) where there is no sample present. Lₘₐₓ is the maximum breast thickness and L_{s-d} is the source-to-detector distance, both as recorded in the DICOM metadata. min(_{\hat{F}(x,y)}) indicates the minimum value of the estimated adipose-equivalent intensity image, which corresponds to the location where breast has been estimated to be the thickest.

In certain aspects, determining breast composition includes estimating breast thickness. According to certain embodiments, once the normalization coefficients c₁ and c₂ are computed, the total breast thickness is estimated pixel-by-pixel in a similar fashion as described in equation (2), assuming that the values indicated in \hat{F}(x,y) correspond to attenuation produced mainly by fatty tissue. This is generated by solving the following expression for \hat{L}(x,y), which can be done using a non-linear optimization technique²¹:

$$\hat{F} ( x , y ) = {c}_{1} ( ( {\int }_{0}^{{ɛ}_{max}} \hat{S} ( ɛ ) {e}^{- {μ}_{ad} ( ɛ ) \hat{L} ( x , y )} \partial ɛ / {\int }_{0}^{{ɛ}_{max}} \hat{S} ( ɛ ) \partial ɛ ) + {c}_{2} ) \qquad ( 4 )$$

Variations and modifications to the approaches described herein may be made. For example, rather than using a simulation of X-ray system source spectra, the methods may include direct measurement of the system spectra at different system settings in a calibration process, which may result in more accurate density measurements. Moreover, rather than assuming a linear response to intensity values from the detector, non-linear assumptions are also possible, as is direct measurement of the detector response to intensity values in a calibration process, which may result in more accurate density measurements.

With respect to equations (2) and (4), rather than solving using a constrained non-linear optimization technique (which computational time requirements can be high considering the large number of pixels in raw digital mammograms), a faster solution may be found using Look-Up-Tables (LUT) for a set of repeated given system specifications. The LUT can be computed for a particular set of system settings in a calibration process, and the only variable inputs in the expression shown in equation (2) will be breast thickness and recorded intensity, with the outputs being ratio of adipose and dense tissue. The expression could then be solved with a LUT by direct 2-to-2 mapping. The same can be said for the expression in equation (4) with a LUT indicating a 1-to-1 mapping from adipose-equivalent intensity values to breast thickness. The expression in equation (2) could be modified to also consider the presence of other tissue types, such as the attenuation produced by skin, or possible masses or calcifications.

Regarding direct pixel-by-pixel estimation of total breast thickness within the mammograms, other estimation methods or direct measurement of pixel-by-pixel total breast thickness could also be applied to the total breast thickness variable used in the expression shown in equation (2). Moreover, the methods of the present disclosure could be also be modified for use in systems employing dual-energy sources, potentially resulting in more precise discrimination of a larger number of different tissue types.

### Systems

Aspects of the present disclosure include systems. According to certain embodiments, the systems find use in practicing the methods of the present disclosure. For example, a system of the present disclosure may be adapted to perform any of the steps described above in the section relating to the methods of the present disclosure.

In certain aspects, provided is a polychromatic absorptiometry system, which system includes a processor and a non-transitory computer readable medium. The non-transitory computer readable medium includes instructions that cause the processor to acquire a raw intensity image of a tissue (e.g., breast tissue) that includes dense tissue and adipose tissue, where the image is generated using a polychromatic electromagnetic radiation source. The instructions further cause the processor to directly measure the proportion of dense tissue and adipose tissue for each pixel of the raw intensity image, assign a value to each pixel based on the directly measured proportion of dense tissue and adipose tissue, and determine tissue composition based on the assigned value of each pixel.

The computer-readable medium (or processor-readable medium) is non-transitory in the sense that it does not include transitory propagating signals per se (e.g., a propagating electromagnetic wave carrying information on a transmission medium such as space or a cable). The media and instructions may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

According to certain embodiments, the system further includes a polychromatic electromagnetic radiation source and a detector adapted to generate the raw intensity image. The polychromatic electromagnetic radiation source may be, e.g., a polychromatic X-ray source.

The systems of the present disclosure may include a display (e.g., an LCD, LED, or other suitable display). In certain aspects, the instructions further cause the processor to display the tissue composition in the form of a dense volume image, a ratio of dense-to-adipose tissue image, or both.

According to certain embodiments, the instructions may further cause the processor to determine the risk of cancer in the tissue.

### Utility

The methods and systems of the present disclosure find use in a variety of applications, including any application in which it is desirable to determine the composition of a tissue (e.g., breast tissue). Applications of interest include, e.g., research applications and clinical applications, e.g., clinical diagnostic applications.

According to certain embodiments, the methods and systems find use in determining breast tissue composition. The semi-automated, area-based Cumulus approach has been reported to have the strongest predictive ability of the existing methods. However, this approach is limited by intra- and inter-reader variability in establishing a threshold for differentiating breast density from surrounding fatty tissue. There have been efforts in automating the threshold selection; however, apart from needing a trained specialist, Cumulus only provides a “masked image,” where image pixels are either categorized as predominantly dense or non-dense tissue. In contrast, the methods of the present disclosure may be fully automated and provide direct measurements of breast-to-adipose tissue ratio, total breast volume, and dense tissue volume in a continuous scale at each image pixel. As demonstrated in the Examples section below, embodiments of the methods also provide measurements of total dense volume and volume percent density with higher association with breast cancer risk than those provided by existing methods, including the Cumulus method.

Existing automated methods that provide volumetric estimates of dense tissue are the Volpara method, the single energy X-ray absorptiometry method (SXA), and the Quantra method, although they have shown less predictive performance than Cumulus. The present methods differ from the Volpara and Quantra approaches in that such methods employ effective linear attenuation coefficients (singular constant value instead or energy-dependent values) derived from the imaging properties and a singular value of breast thickness. These assumptions simplify the expressions to be solved but may reduce the accuracy of the measurements and their utility in cancer risk discrimination. Embodiments of the methods of the present disclosure consider the dependency of attenuation coefficients with energy, solving the resulting more complicated expressions using computerized optimization techniques. The methods of the present disclosure may also consider breast density relative to breast thickness at each particular location, employing the adipose-equivalent image, instead of the value of a single fatty tissue pixel used in previous methods. As an advantage over single X-ray absorptiometry (SXA; see, e.g., Shepherd et al. (2005) Technology in Cancer Research and Treatment 4(2):173-182) (cited above), the methods of the present disclosure consider the polychromatic nature of the mammography system source, providing better estimates of the amount of dense tissue. The methods of the present disclosure also do not require a phantom present in the acquired images, in contrast with SXA and other previous methods, facilitating the analysis of retrospective data or new data where a phantom is not present.

Moreover, embodiments of the methods of the present disclosure provide estimates of dense tissue volume and dense-to-adipose tissue ratio that yield stronger associations with breast cancer risk, presenting a superior ability to predict future breast cancer occurrences and stratify women according to cancer risk. In certain aspects, the methods find use in image pre-processing to improve display and/or visualization of density and masses.

The following examples are offered by way of illustration and not by way of limitation.

### Experimental

### EXAMPLE 1

### Density Estimation

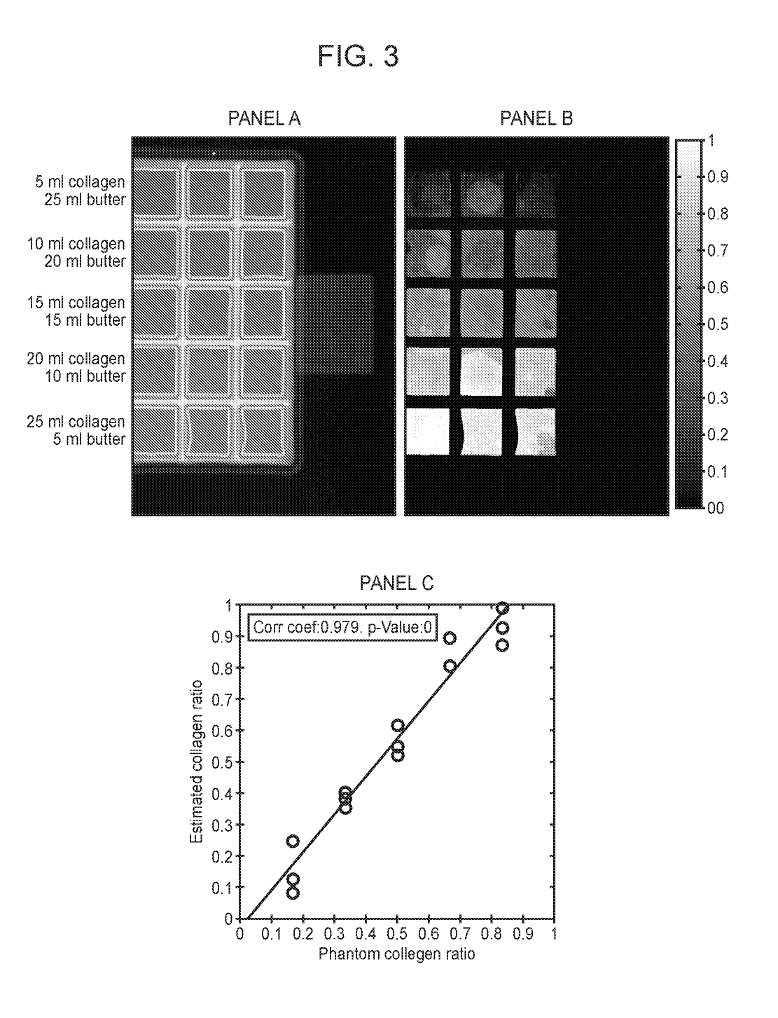

A density estimation method was preliminarily evaluated in a digital mammography image of a physical phantom with known characteristics. This physical phantom was fabricated by filling 15 wells of a silicone ice cube tray with different concentrations of butter and collagen. The example mammogram of this phantom is shown in FIG. 3, Panel A. The location of the wells within the image, indicated by the outlines, was determined automatically using image processing techniques (thresholding and morphological operations). The height of the sample was 35 mm, the silicone mold thickness was 3 mm for all wells, and each well was also topped with a 4 mm layer of paraffin. Five different conditions of collagen vs. butter concentrations were performed in triplicate, with increasing value of collagen concentration: 5 ml collagen vs. 25 ml butter, 10 ml collagen vs. 20 ml butter, 15 ml collagen vs. 15 ml butter, 20 ml collagen vs. 10 ml butter, and 25 ml collagen vs. 5 ml butter, for rows 1 to 5, respectively.

The PXA technique was used to estimate the volume of collagen and ratio of collagen vs. total volume for each pixel in the phantom wells (collagen density ratio). FIG. 3, Panel B, shows the estimated collagen density ratio image. It was considered that butter replicated the attenuation properties of adipose tissue and collagen replicated the attenuation properties of dense tissue. In this case, equations (2), (3) and (4) were modified to consider a constant layer of silicone of known thickness (3mm) related to the mold thickness and a constant layer of paraffin of known thickness (4mm) related to each well lid.

Table 1 summarizes the average density estimations for each well in the phantom, averaged throughout the pixels on each well. Density was quantified as the rate of collagen volume per total volume. The correlation of the fabricated collagen density in the phantom with the estimated values was analyzed using a Pearson's linear approach, resulting on a very high correlation coefficient of 0.979, which was significant (p<10⁻⁹). A plot of the estimated collagen density values displayed against the actual fabricated values together with their correlation coefficient is shown in FIG. 3, Panel C. A very strong correlation between the fabricated and estimated density values was observed. A slight offset in the estimation values can be derived from the consideration of adipose and dense breast tissue attenuation values instead of those for butter and collagen, which was employed for simplicity. The variability of the estimated measurements in each tested collagen/butter concentration condition may be derived from the physical variability introduced during the fabrication process.

TABLE 1
Estimated average collagen density ratio throughout each well

| Fabricated | Estimated density | Estimated density | Estimated density |
|---|---|---|---|
| density | well #1 | well #2 | well #3 |
| 0.167 | 0.127 | 0.248 | 0.083 |
| 0.333 | 0.403 | 0.384 | 0.352 |
| 0.5 | 0.618 | 0.549 | 0.523 |
| 0.667 | 0.807 | 0.894 | 0.805 |
| 0.833 | 0.989 | 0.928 | 0.871 |

### EXAMPLE 2

### Association with Cancer Risk

The PXA method was evaluated in 131 mammograms from unaffected breasts prior to a cancer diagnosis in the contralateral breast (cases) and 239 mammograms from healthy women without breast cancer (controls). Control women were chosen to match the case patients by age and race. Patient demographics are summarized in Table 2. The study protocol was approved by the Stanford University Institutional Review Board. All images were acquired as part of the clinical standard for screening mammography, comprising two views of each breast, cranio-caudal (CC) and medio-lateral oblique (MLO) views. The CC view of the non-cancerous breast in cases and the corresponding CC view for the matched control were used as study images. All mammograms were acquired with either a General Electric (GE) Senograph Essential FFDM unit or Senograph 2000D (General Electric Medical Systems, Milwaukee, Wis., USA). The system produces raw intensity images with 14-bit dynamic range for each pixel (values ranging from 0 to 16383).

TABLE 2
Distribution of patient characteristics in case and control women

| | | Control mean | | |
|---|---|---|---|---|
| | Case mean (SD) | (SD) of % | Total mean (SD) | q- |
| Characteristic | or % (n = 131) | (n = 239) | or % (n = 370) | value |
| Age (years) | 54.35 (10.90) | 55.03 (11.04) | 54.79 (10.98) | 0.35 |
| BMI (kg/m²) | 26.54 (6.21) | 25.64 (5.87) | 25.96 (6.00) | 0.21 |
| White (%) | 62.83 | 62.76 | 62.43 | 0.44 |
| Black (%) | 6.87 | 5.02 | 5.68 | 0.31 |
| Asian (%) | 22.90 | 23.43 | 23.24 | 0.44 |
| Other (%) | 8.40 | 8.79 | 8.65 | 0.44 |
| Premenopausal (%) | 25.95 | 24.27 | 24.86 | 0.40 |
| Perimenopausal (%) | 3.82 | 9.62 | 7.57 | 0.07 |
| Postmenopausal (%) | 70.23 | 66.11 | 67.57 | 0.31 |
| Cumulus-DA (cm²) | 33.68 (19.74) | 28.72 (17.41) | 30.47 (18.39) | 0.03 |
| Cumulus-PD (%) | 27.44 (15.10) | 25.35 (15.12) | 26.09 (15.13) | 0.22 |
| Volpara-DV (cm³) | 66.82 (41.58) | 56.86 (31.71) | 60.38 (35.78) | 0.03 |
| Volpara-VPD (%) | 11.85 (8.41) | 11.22 (7.79) | 11.44 (8.01) | 0.31 |
| PXA DV (cm³) | 95.32 (60.13) | 76.14 (51.05) | 82.93 (55.13) | 0.01 |
| PXA VPD (%) | 14.39 (8.92) | 12.41 (6.80) | 13.11 (7.66) | 0.03 |

FIG. 4 displays examples of percentage of dense-to-adipose tissue images as generated by our PXA method for case and control mammograms, displayed alongside the original mammogram.

Cumulus (v6) dense area (DA) and percent density (PD), as well as Volpara (v150, Matakina) dense volume (DV) and volumetric percent density (VPD) values were collected by an expert user of both software applications. Two overall measurements were computed from the pixel-by-pixel images produced by our PXA method for comparison: (1) PXA-DV, computed by adding pixel by pixel the result from the multiplication of dense ratio and breast thickness; (2) PXA-VPD, computed by averaging the pixel-based dense ratio values throughout the mammogram, weighted by breast thickness.

The distribution of patient demographics, Cumulus-DA, Cumulus-PD, Volpara-DV, Volpara-VPD, PXA-DV, and PXA-VPD measurements in case and control women were compared using a two-sided t-test. The resulting p-values were later corrected using a multiple hypothesis testing analysis by estimating the false positive discovery rate q-values, considering all tested measurements. The associations of each quantitative measurement with breast cancer risk was studied by computing their odds ratios (OR) as quartiles (defined among controls) and as continuous variables (in standard deviation (SD) increments), both unadjusted and adjusted for age, race, body-mass index, and menopausal status. The area under the curve (AUC) of a receiver operating characteristic (ROC) curve was also computed for each method to compare their ability to discriminate between cases and controls, both unadjusted and adjusted for age, race, body-mass index, and menopausal status.

The mean and SD values for the demographics and each quantitative measurement evaluated for the case and control mammograms are summarized in Table 2. The false-positive corrected two-sided t-test comparing the distribution differences between case and control women indicated that the absolute measurements (dense area or volume) for the three methods evaluated (Cumulus, Volpara and PXA) showed statistically significant differences (under a q<0.05 criterion) between control and case patients. However, only the PXA quantification measurements were statistically significant for the relative density measurements (PD or VPD) under a q<0.05 criterion.

Table 3 summarizes the ORs estimated for the Cumulus, Volpara and PXA measurements as quartiles and as continuous variables, both unadjusted and adjusted by age, race, body-mass index, and menopausal status. Overall, measurements made by all methods were positively associated with breast cancer risk, with higher associations observed for the PXA method.

TABLE 3
Distribution of patient characteristics in case and control women

| | | | Quart. | Quart. | SD | SD | | |
|---|---|---|---|---|---|---|---|---|
| | | | unadjust. | adjust.* | unadjust. | adjust.* | | Adjust.* |
| Measurement | No. | No. | OR | OR | OR | OR | AUC | AUC |
| Quartile | Cases | Controls | (95% CI) | (95% CI) | (95% CI) | (95% CI) | (95% CI) | (95% CI) |
| Cumulus-DA | 131 | 239 | — | — | 1.31 | 1.38 | 0.56 | 0.63 |
| | | | | | (1.05, 1.71) | (1.08, 1.76) | (0.50, 0.62) | (0.57, 0.69) |
| Q1 (0.96) | 16 | 60 | 1.00 (Ref.) | 1.00 (Ref.) | — | — | — | — |
| Q2 (14.22) | 43 | 59 | 2.73 | 3.7 | — | — | — | — |
| | | | (1.39, 5.38) | (1.70, 8.07) | | | | |
| Q3 (27.61) | 32 | 60 | 2 (0.99, 4.02) | 2.71 | — | — | — | — |
| | | | | (1.16, 6.32) | | | | |
| Q4 (40.12) | 40 | 60 | 2.5 | 2.84 | — | — | — | — |
| | | | (1.26, 4.94) | (1.20, 6.73) | | | | |
| Cumulus-PD | 131 | 239 | | | 1.15 | 1.40 | 0.54 | 0.63 |
| | | | | | (0.92, 1.43) | (1.04, 1.90) | (0.48, 0.60) | (0.57, 0.69) |
| Q1 (0.59) | 26 | 60 | 1.00 (Ref.) | 1.00 (Ref.) | — | — | — | — |
| Q2 (12.04) | 30 | 59 | 1.17 | 1.54 | — | — | — | — |
| | | | (0.62, 2.22) | (0.74, 3.21) | | | | |
| Q3 (25.96) | 39 | 60 | 1.5 | 2.24 | — | — | — | — |
| | | | (0.81, 2.8) | (0.95, 5.27) | | | | |
| Q4 (36.36) | 36 | 60 | 1.38 | 2.86 | — | — | — | — |
| | | | (0.75, 2.57) | (1.08, 7.58) | | | | |
| Volpara-DV | 131 | 239 | | | 1.31 | 1.36 | 0.56 | 0.63 |
| | | | | | (1.06, 1.63) | (1.07, 1.72) | (0.50, 0.62) | (0.57, 0.69) |
| Q1 (13.31) | 25 | 60 | 1.00 (Ref.) | 1.00 (Ref.) | — | — | — | — |
| Q2 (35.06) | 35 | 59 | 1.42 | 1.40 | — | — | — | — |
| | | | (0.76, 2.66) | (0.73, 2.69) | | | | |
| Q3 (50.14) | 30 | 60 | 1.2 | 1.57 | — | — | — | — |
| | | | (0.63, 2.28) | (0.75, 3.29) | | | | |
| Q4 (73.33) | 41 | 60 | 1.64 | 1.45 | — | — | — | — |
| | | | (0.89, 3.026) | (0.69, 3.04) | | | | |
| Volpara-VPD | 131 | 239 | | | 1.08 | 1.27 | 0.52 | 0.62 |
| | | | | | (0.87, 1.34) | (0.95, 1.70) | (0.46, 0.58) | (0.56, 0.68) |
| Q1 (1.76) | 33 | 59 | 1.00 (Ref.) | 1.00 (Ref.) | — | — | — | — |
| Q2 (5.19) | 29 | 60 | 0.86(0.47, 1.60) | 1.14 | — | — | — | — |
| | | | | (0.53, 2.46) | | | | |
| Q3 (9.06) | 38 | 60 | 1.13 | 1.16 | — | — | — | — |
| | | | (0.63, 2.04) | (0.51, 2.62) | | | | |
| Q4 (15.09) | 31 | 60 | 0.92 | 1.12 | — | — | — | — |
| | | | (0.50, 1.70) | (0.36, 3.47) | | | | |
| PXA-DV | 131 | 239 | | | 1.41 | 1.41 | 0.60 | 0.64 |
| | | | | | (1.13, 1.75) | (1.11, 1.81) | (0.54, 0.66) | (0.58, 0.70) |
| Q1 (4.10) | 18 | 60 | 1.00 (Ref.) | 1.00 (Ref) | — | — | — | — |
| Q2 (42.42) | 32 | 59 | 1.81 | 1.75 | — | — | — | — |
| | | | (0.92, 3.57) | (0.84, 3.65) | | | | |
| Q3 (65.88) | 32 | 60 | 1.78 | 1.82 | — | — | — | — |
| | | | (0.90, 3.51) | (0.83, 4.00) | | | | |
| Q4 (97.23) | 49 | 60 | 2.72 | 3.26 | — | — | — | — |
| | | | (1.42, 5.20) | (1.47, 7.20) | | | | |
| PXA VPD | 131 | 239 | | | 1.29 | 1.53 | 0.56 | 0.65 |
| | | | | | (1.04, 1.59) | (1.17, 2.00) | (0.49, 0.62) | (0.59, 0.70) |
| Q1 (4.07) | 24 | 60 | 1.00 (Ref.) | 1.00 (Ref) | — | — | — | — |
| Q2 (7.45) | 35 | 59 | 1.48 | 1.98 | — | — | — | — |
| | | | (0.79, 2.79) | (0.97, 4.04) | | | | |
| Q3 (10.54) | 27 | 60 | 1.12 | 1.32 | — | — | — | — |
| | | | (0.58, 2.17) | (0.60, 2.92) | | | | |
| | | | 1.88 | 5.30 | | | | |
| Q4 (15.47) | 45 | 60 | (1.02, 3.45) | (1.99, 14.16) | — | — | — | — |

Considering the adjusted ORs, the strongest continuous association observed for each SD increment was observed for PXA-VPD (OR=1.53 (1.17, 2.00)). PXA-VPD also presented the highest association with cancer risk for the top quartile, presenting 5.30 (1.99, 14.16) times the risk of women in the bottom quartile, compared to 2.86 (1.08, 7.58) for Cumulus-PD and 1.12 (0.36, 3.47) for Volpara-VPD. Absolute measurements of dense area or volume also were more highly associated with cancer risk when measured using the PXA method, showing an OR of 3.26 (1.47, 7.20) for women on top vs. bottom quartile for PXA-DV compared to 2.84 (1.20, 6.73) and 1.45 (0.69, 3.04) produced by Cumulus-PD and Volpara-VPD, respectively.

PXA measurements also showed a greater ability to discriminate between cases and controls in terms of AUC. The adjusted AUC values were low (0.65 (0.59, 0.70) for PXA-VPD and 0.64 (0.58, 0.70) for PXA-DV), but similar to that previously reported by others, indicating its limited value in individual cancer risk prediction. Higher differences between the evaluated methods in terms of AUC values were observed when no adjustment by other factors (age, race, body-mass index, and menopausal status) was made, with PXA-DV presenting higher values than the rest. This may highlight the contribution presented by these other factors in the discrimination of cases and controls, the possible higher correlation of PXA-DV with any of these other factors than that of PXA-VPD, and the higher performance of PXA-DV when used as an independent predictor.

The PXA method presented here appears to be a valid automated alternative to the labor-intensive semi-automated Cumulus approach for quantifying breast density when raw FFDM images are available for analysis, and it also offers the possibility of pixel-by-pixel analysis of volume-based methods, while increasing the association of overall quantifications with cancer risk. These quantifications, alone or jointly with other risk factors, might be useful to stratify women in the population according to risk for tailored screening or interventions.

### Conclusion

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A computer-implemented method of determining areas of tissue density and composition by use of polychromatic absorptiometry, comprising:
(a) acquiring a raw, digital intensity image of a tissue comprising different areas of tissue density, wherein less dense tissue comprises adipose tissue, and wherein the image is generated using a polychromatic electromagnetic radiation source;
(b) correcting attenuation effects on density associated with energy differences within the polychromatic electromagnetic radiation source;
(c) directly measuring the proportion of dense tissue and adipose tissue for each pixel of the raw intensity image using an adipose-equivalent intensity estimation;
(d) creating an assigned value to each pixel of step (c) based on the directly measured proportion of dense tissue and adipose tissue; and
(e) determining tissue composition based on the assigned value of each pixel.

2. The method according to claim 1, further comprising a step of irradiating tissue in vivo using the polychromatic electromagnetic radiation source to generate the raw intensity image.

3. The method according to claim 1 or claim 2, wherein the polychromatic electromagnetic radiation source is a polychromatic X-ray source.

4. The method according to any one of claims 1 to 3, further comprising a step of displaying the tissue composition in the form of a dense volume image, a ratio of dense-to-adipose tissue image, or both.

5. The method according to any one of claims 1 to 4, further comprising a step of determining the risk of cancer in the tissue based on the determined tissue composition.

6. The method according to any one of claims 1 to 5, wherein the tissue is breast tissue.

7. The method according to claim 1, wherein the raw, digital intensity image comprises a single digital mammography screening image; the step of correcting attenuation effects produces and adipose-equivalent estimation of the raw, digital image intensity; and the correcting attenuation also produces a breast thickness estimation.

8. The method according to claim 1, wherein the raw, digital intensity image is an X-ray mammogram image, the step of determining tissue composition comprises producing a quantification of dense volume of an imaged breast and also producing a ratio of dense to adipose tissue of the imaged breast.

9. The method according of claim 8, further comprising a step of calculating a risk of developing breast cancer in the imaged breast, based on the quantification of dense volume in the imaged breast and the ratio of dense-to-adipose tissue in the imaged breast.

10. A polychromatic absorptiometry system, comprising:
a processor;
a non-transitory computer readable medium comprising instructions that cause the processor to:
acquire a raw intensity image of a tissue comprising dense tissue and adipose tissue, wherein the image is digital and generated using a polychromatic electromagnetic radiation source;
directly measure the proportion of dense tissue and adipose tissue for each pixel of the raw intensity image, wherein density is calculated using a correction for energy variations within the polychromatic electromagnetic radiation source;
assign a value to each pixel based on the directly measured proportion of dense tissue and adipose tissue; and
determine tissue composition based on the assigned value of each pixel.

11. The system of claim 10, further comprising a polychromatic electromagnetic radiation source and a detector adapted to generate the raw intensity image.

12. The system of claim 10, wherein the polychromatic electromagnetic radiation source is a polychromatic X-ray source.

13. The system of claim 10, further comprising a display that graphically displays areas of density within the tissue.

14. The system of claim 10, wherein the instructions further cause the processor to display the tissue composition in the form of a dense volume image, a ratio of dense-to-adipose tissue image, or both.

15. The system of any one of claims 10-14, wherein the instructions further cause the processor to determine the risk of cancer in the tissue.
